(19) United States
(12) Patent Application Publication
(10) Pub. No.: US 2025/0135374 A1
(43) Pub. Date: May 1, 2025

(54) Method for producing mixtures containing 2-(2-hydroxyethyl)-piperidinyl carbamide acid secondary butyl ester

(71) Applicant: Saltigo GmbH, Leverkusen (DE)

(72) Inventor: Reinhard Langer, Toenisvorst (DE)

(73) Assignee: Saltigo GmbH, Leverkusen (DE)

(21) Appl. No.: 18/687,697

(22) PCT Filed: Aug. 29, 2022
(86) PCT No.: PCT/EP2022/073974

§ 371 Date: Feb. 28, 2024

(30) Foreign Application Priority Data
Aug. 30, 2021 (EP) 21193854.3

(51) Int. Cl. B01D 3/14; B01D 1/22

(52) U.S. Cl. CPC B01D 3/148; B01D 1/223

(57) ABSTRACT

The invention relates to an improved process for producing mixtures comprising sec-butyl 2-(2-hydroxyethyl)piperidinylcarbamate from a crude product by thermal treatment with short residence times, apparatuses for this process, the use of such apparatuses for such processes, and the mixtures of the invention obtainable from the process.

The invention relates to an improved process for producing mixtures comprising sec-butyl 2-(2-hydroxyethyl)piperidinylcarbamate from a crude product by thermal treatment with short residence times, apparatuses for this process, the use of such apparatuses for such processes, and the mixtures of the invention obtainable from the process.

sec-Butyl 2-(2-hydroxyethyl)piperidinylcarbamate (also referred to as 1-piperidinecarboxylic acid 2-(2-hydroxyethyl)-1-methylpropyl ester; CAS No. 119515-38-7, INN: icaridin, picaridin, Saltidin®, commercial product: Saltidin® from Saltigo GmbH, Leverkusen, Germany) has the following structure of the formula (I):

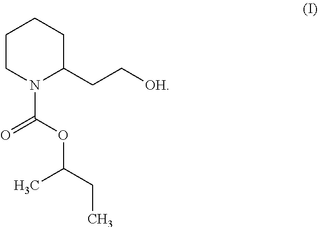 [SMILES: CCC(C)OC(=O)N1CCCCC1CCO]

The molecule contains 2 stereocenters. Formula (I), according to the invention, encompasses each of the individual 4 possible stereoisomers, and also any mixtures and the racemates, i.e. the mixtures of the individual or of the two possible enantiomer pairs in equal parts.

This compound is a highly effective and well tolerated insect repellent against mosquitoes and ticks which is commercially available in various formulations for application to the human or animal skin.

The preparation of sec-butyl 2-(2-hydroxyethyl)piperidinylcarbamate is described, for example, in EP 0 537 534 A1, according to which sec-butyl 2-(2-hydroxyethyl)piperidinylcarbamate is obtained in yields of 89 percent of theory (based on 2-(2-hydroxyethyl)piperidine). In the last stage, 2-(2-hydroxyethyl)piperidine is chemically reacted with sec-butyl chloroformate in the presence of immiscible solvents and base. The reaction gives rise to the desired product alongside, for example, sodium chloride and organic secondary components, some of which are colored and/or may have an unpleasant odor. For example, EP 0 537 534 A1 states that crude products from this reaction are often contaminated and therefore have to be purified by distillation or by chromatography, which can also lead to considerable yield losses in addition to the increased cost and inconvenience. Furthermore, it is stated therein that the main side reaction results in formation of products that derive from a competing reaction of the chloroformic ester with the hydroxyl group from the 2-(2-hydroxyethyl)piperidine.

For instance, by the customary known production process, the compound of the formula (II)

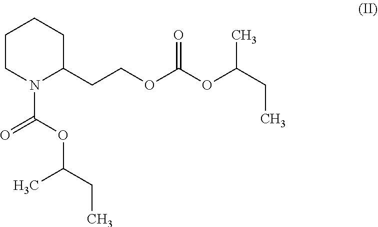 [SMILES: CCC(C)OC(=O)OCCC1CCCCN1C(=O)OC(C)CC]

is present in a content of about 1% by weight or more. In addition, the mixtures produced by the customary processes that contain more than 90% by weight of compound of the formula (I) have a Hazen color number of more than 40.

In the distillative purification, it is also possible for breakdown reactions to occur at low pressures, the malodorous products of which contaminate the distillate. It has therefore not been possible to date to successfully use distillation as the purification method for sec-butyl 2-(2-hydroxyethyl)piperidinylcarbamate on an industrial scale.

Typically, the reaction mixture present after the reaction is therefore advantageously subjected to aqueous workup, in which case it is necessary to neutralize any base present by addition of acid. This is typically followed by the removal of the organic phase, containing the desired product and organic secondary components, and the water-immiscible organic solvent used in the reaction, for example hexane or toluene. It has been found that, on an industrial scale, in which the product is produced in batch sizes of more than 1000 kg, several washes of the organic phase thus obtained with water and/or acid have to be conducted in order to obtain a product suitable for the production of insect repellent formulations, which meets the requirement on purity, color and olfactory quality. After the washes, the solvent is typically distilled out of the organic phase under reduced pressure, leaving the product and all or some of the organic and inorganic secondary components/impurities in the bottoms. The bottoms are then withdrawn from the reactor and filtered, and constitute the finished end product. In the process conducted to date on an industrial scale, the sec-butyl 2-(2-hydroxyethyl)piperidinylcarbamate product is obtained in this way in yields of about 90 percent of theory based on 2-(2-hydroxyethyl)piperidine), in purities of 97.0 to 98.5 percent by weight. It typically has a Hazen color number of 40 or more and a slight characteristic unpleasant odor. Even though sec-butyl 2-(2-hydroxyethyl)piperidinylcarbamate is obtainable in very good yields and purities even on an industrial scale by this currently best process, the prior art processes nevertheless have the disadvantage that the workup of the crude product is very complex and energy-intensive, produces a large amount of waste and hence incurs high costs.

However, the prior art also discloses syntheses of sec-butyl 2-(2-hydroxyethyl)piperidinylcarbamate in which the reaction of 2-(2-hydroxyethyl)piperidine with sec-butyl chloroformate in the presence of solvent is followed by several extractive washes of the organic phase and, as the last step, distillation of the product under reduced pressure.

For instance, CN 102167681 A1 describes the reaction of 2-(2-hydroxyethyl)piperidine with sec-butyl chloroformate in the presence of solvents such as dichloromethane, chloroform, benzene, toluene, tetrahydrofuran, n-hexane, cyclohexane or methylcyclohexane as solvents. Bases used are organic bases such as 2-(2-hydroxyethyl)piperidine, triethylamine, pyridine, or trimethylamine. The organic phase containing the crude product was repeatedly washed with water and sodium chloride solution.

Subsequently, the organic phase was dried with sodium sulfate or magnesium sulfate and filtered, and the solvent was distilled off. The residue was then distilled, giving the product as distillate with a boiling point of 140 to 152° C. at 5 mm Hg (6.67 hPa). With batch sizes of about 20 kg of product, the product by this process was obtained in yields of 84 to 87 percent of theory and with a content of 97.5 to 98.0 area percent (ascertained via gas chromatography).

However, in-house experiments (see examples) have shown that, in the distillation of a crude product that was likewise obtained after several aqueous washes, in the case of distillation under reduced pressure at 20 hPa and bottom temperature about 175° C., significant breakdown of the product occurred, in which 2-butanol was eliminated and the cyclic carbamate of 2-(2-hydroxyethyl)piperidine of the formula (III):

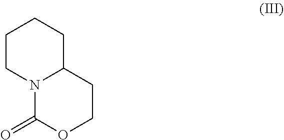 [SMILES: O=C1OCCC2CCCCN12]

was formed, with condensation of the butanol not in the condenser, in which the product of the formula (I) typically condenses, but only in a downstream cold trap, leaving the carbamate in the bottoms. Since it is virtually impossible to perform a batchwise distillation at below 10 hPa on an industrial scale, a workup according to CN 102167681 A1 is not an option for this purpose.

Moreover, the products obtainable on industrial scales to date, containing more than 90% by weight of compound of the formula (I), have a slight but unpleasant odor that can be unpleasant in insect repellent formulations and may need to be masked by fragrances.

The technical problem that thus remained was that of providing a process for preparing sec-butyl 2-(2-hydroxyethyl)piperidinylcarbamate that does not have the disadvantages of the prior art processes. There was therefore a need for an improved process by which sec-butyl 2-(2-hydroxyethyl)piperidinylcarbamate is obtainable on an industrial scale in at least an equal yield and purity or even in a higher yield and/or with a lower level of unwanted secondary components and/or better color quality and/or olfactory quality, but takes less time and energy and produces less waste, and hence the technical problem was that of providing such an improved process.

What has now been found, surprisingly, is an improved process for producing a mixture containing, as components, from 98.8% to 100.0% by weight, preferably from 99.0 to 99.9% by weight, of compound of the formula (I):

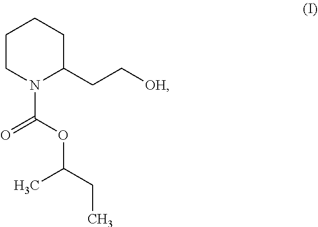 [SMILES: CCC(C)OC(=O)N1CCCCC1CCO]

from 0.00% to 0.70% by weight, preferably from 0.0001% to 0.20% by weight, of compound of the formula (II):

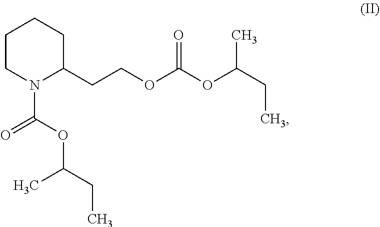 [SMILES: CCC(C)OC(=O)OCCC1CCCCN1C(=O)OC(C)CC]

and further constituents, where the percentages by weight of the components add up to 100% by weight, where the mixture has a Hazen color number of 0 to 15 measured by the DIN EN ISO 6271 method,
proceeding from a crude product containing
   from 94% to 99.0% by weight of compound of the formula (I) and
   from 0.50% to 5.0% by weight of compound of the formula (II),
wherein at least a fraction A (distillate, mixture of the invention) is separated from a fraction B (bottoms) and a gaseous fraction C, by
   a) subjecting the crude product in the form of a liquid phase to thermal treatment, such that a gas stream G comprising fraction A and fraction C is produced, and fraction B remains as a liquid phase, and
   b) simultaneously and/or successively discharging fraction B from the process in liquid form, and
   c) simultaneously and/or successively condensing fraction A out of gas stream G and discharging it from the process, giving the mixture of the invention, and
   d) optionally simultaneously and/or successively condensing fraction C and discharging it from the process,
wherein the residence time of the liquid phase during the thermal treatment, preferably the residence time of the liquid phase containing a crude product and/or fraction B or any mixtures thereof, is from 1 to 900 seconds, preferably from 10 to 600 seconds, more preferably from 10 to 300 seconds.

What has also been found is a further embodiment of the process of the invention for producing a mixture containing, as components, from 98.8% to 100.0% by weight, preferably from 99.0 to 99.9% by weight, of compound of the formula (I):

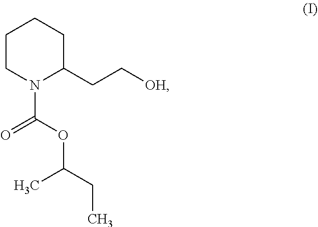 [SMILES: CCC(C)OC(=O)N1CCCCC1CCO]

from 0.00% to 0.70% by weight, preferably from 0.00% to 0.6% by weight, further preferably from 0.00% to 0.5% by weight, especially preferably from 0.0001% to 0.20% by weight, of compound of the formula (II),

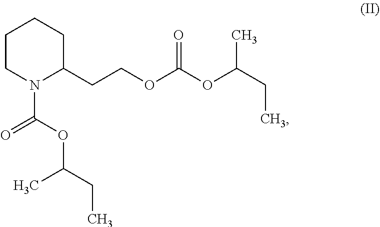 [SMILES: CCC(C)OC(=O)OCCC1CCCCN1C(=O)OC(C)CC]

and further constituents, where the percentages by weight of the components add up to 100% by weight, where the mixture has a Hazen color number of 0 to 15 measured by the DIN EN ISO 6271 method,
proceeding from a crude product containing
   from 94% to 98.0% by weight of compound of the formula (I) and
wherein at least a fraction A (distillate, mixture of the invention) is separated from a fraction B (bottoms) and a gaseous fraction C, by
   a) subjecting the crude product in the form of a liquid phase to thermal treatment, such that a gas stream G comprising fraction A and fraction C is produced, and fraction B remains as a liquid phase, and
   b) simultaneously and/or successively discharging fraction B from the process in liquid form, and
   c) simultaneously and/or successively condensing fraction A out of gas stream G and discharging it from the process, giving the mixture of the invention, and
   d) optionally simultaneously and/or successively condensing fraction C and discharging it from the process,
wherein the residence time of the liquid phase during the thermal treatment, preferably the residence time of the liquid phase containing a crude product and/or fraction B or any mixtures thereof, is from 1 to 900 seconds, preferably from 10 to 600 seconds, more preferably from 10 to 300 seconds.

It is preferably also possible by the process of the invention to produce mixtures containing, as components, from 98.8% to 100.0% by weight, preferably from 99.0% to 99.9% by weight, of compound of the formula (I), from 0.00% to 0.70% by weight, preferably from 0.0001% to 0.20% by weight, of compound of the formula (II), from 0.0001% to 2.0% by weight of compound of the formula (III), and further constituents, where the proportions by weight of the components add up to 100% by weight,

wherein the mixture has a Hazen color number of 0 to 15, measured by the DIN EN ISO 6271 method.

In a further embodiment, it is preferably also possible by the process of the invention to produce mixtures containing, as components, from 98.8% to 100.0% by weight, preferably from 99.0% to 99.9% by weight, of compound of the formula (I), from 0.00% to 0.60% by weight, preferably from 0.0001% to 0.20% by weight, of compound of the formula (II), from 0.0001% to 2.0% by weight of compound of the formula (III), and further constituents, where the proportions by weight of the components add up to 100% by weight,

wherein the mixture has a Hazen color number of 0 to 15, measured by the DIN EN ISO 6271 method.

In a further embodiment, it is preferably also possible by the process of the invention to produce mixtures containing, as components, from 98.8% to 100.0% by weight, preferably from 99.0% to 99.9% by weight, of compound of the formula (I), from 0.00% to 0.50% by weight, preferably from 0.0001% to 0.20% by weight, of compound of the formula (II), from 0.0001% to 2.0% by weight of compound of the formula (III), and further constituents, where the proportions by weight of the components add up to 100% by weight,

wherein the mixture has a Hazen color number of 0 to 15, measured by the DIN EN ISO 6271 method.

In the process of the invention, a crude product that results from the chemical production process and subsequent, typically extractive, purification steps is used.

Preferably, in the process of the invention, a crude product containing from 94.0% to 99.0% by weight of compound of the formula (I) and from 0.50% to 5.0% by weight of compound of the formula (II) and having a Hazen color number of at least 40, measured by the DIN EN ISO 6271 method, is used.

Likewise preferably, in the process of the invention, a crude product containing from 94.0% to 98.0% by weight of compound of the formula (I) and having a Hazen color number of at least 40, measured by the DIN EN ISO 6271 method, is used.

For example, the crude product is produced by a method comprising
i. reacting 2-hydroxyethylpiperidine with sec-butyl chloroformate in the presence or absence of at least one base and in the presence or absence of at least one solvent, giving a reaction mixture containing from 35% to 65% by weight of compound of the formula (I), and
ii. subsequently, optionally while mixing, adding acid and/or water to the reaction mixture from step i., forming a biphasic reaction mixture comprising an organic phase and an aqueous phase, and
iii. subsequently, optionally separating the organic phase from the aqueous phase of the reaction mixture from step ii., and
iv. subsequently, optionally washing the organic phase from step iii. with aqueous acid, and
v. subsequently, optionally drying the organic phase from step iv., and
vi. subsequently, optionally separating the at least one solvent from the organic phase from step v., giving the crude product.

In a preferred embodiment, this crude product can be used in the process of the invention.

In the process of the invention, three different fractions are separated, where fraction A is the mixtures of the invention, a higher-boiling fraction B is a mixture having typically from 40% to 95% by weight of compound of the formula (I) and from 1.5% to 50% by weight of compound of the formula (II), and a lower-boiling fraction C is a mixture having typically from 0.0% to 0.5% by weight of compound of the formula (I) and containing essentially water, butanol and toluene, depending on the quality of the starting material. In the process of the invention, the liquid crude product as liquid phase is subjected to thermal treatment by suitable technical means. It is essential to the process of the invention that the residence time of the liquid phase during the thermal treatment, preferably the residence time of the liquid phase containing a crude product and/or fraction B or any mixtures thereof, is from 1 to 900 seconds, preferably from 10 to 600 seconds, more preferably from 10 to 300 seconds.

It will be apparent that the residence time describes the time from commencement of the thermal treatment of the crude product, i.e. from commencement of the entry of the crude product into the region in which the thermal treatment is effected, through the depletion of the crude product in gas stream G within the region in which the thermal treatment is effected, with formation of the fraction B that then exists, up to the end of the thermal treatment, i.e. up to the juncture at which fraction B leaves the region in which the thermal treatment is effected again.

The thermal treatment of the crude product is preferably effected by contacting the crude product with a fixed heated surface.

According to the invention, the residence time of the liquid phase during the thermal treatment is measured at the temperatures and pressures and with the heat input with which the process of the invention is also conducted. The person skilled in the art is aware of the appropriate methods for this purpose. For example, the residence time can be ascertained by what is called the tracer method. What are typically obtained in these measurements are residence time distribution (RTD) curves. The average residence time can then be calculated by customary methods. The average residence time can also be ascertained with sufficient accuracy by simplified methods. For example, the average residence time T, measured in seconds, for example, in a continuous process in a state of equilibrium is the quotient V/R of the volume V of the liquid phase within the space, measured in cubic centimeters, for example, in which the thermal treatment is being effected, and the inflow rate R of the liquid phase, measured in cubic centimeters per second, for example, into the space in which the thermal treatment is taking place. According to the invention, the residence time is the average residence time calculated from the measured residence time distribution and/or determined by simplified methods.

The process of the invention excludes distillation processes in which the liquid phase is subjected to thermal treatment in an operation such that the entire original liquid phase is heated in a volume, and the volatile components are evaporated out. In these so-called batchwise distillations, the residence time depends on the distillation time. In the case of simultaneous withdrawal of distillate, measured in cubic centimeters per second, for example, the average residence time of the liquid phase in a batchwise distillation corresponds to half the distillation time.

During the preferred process of the invention, the fixed surface is at a temperature sufficient to produce a gas stream G containing fraction A and fraction C. Fraction B is not converted to gas phase at the heated fixed surface, but rather transported away from the fixed heated surface as a liquid film via technical means, generally into a vessel provided for fraction B. Gas stream G has a boiling point of about 330° C. at ambient pressure, a boiling point of 178 to 182° C. at 20 hPa and of 194 to 190° C. at 35 hPa, and at 20 hPa typically has a temperature of 170 to 180° C. measured at the top of the evaporation space.

In the process of the invention, the gas stream G is preferably produced at a temperature of 120 to 200° C., preferably of 120 to 185° C., more preferably of 120 to 175° C., and at a pressure of 1 to 35 hPa, preferably of 1 to 21 hPa, more preferably of 2 to 15 hPa. At higher pressures and the consequently required higher temperatures for production of the gas stream G, there is an increase in the breakdown of the compound of the formula (I). At the pressures mentioned, the temperature for the heated fixed surface is preferably chosen such that it is not more than 40° C., preferably not more than 30° C., above the boiling point of fraction A, i.e. of the mixture of the invention at the respective pressure.

In the process of the invention, fraction A is condensed out of gas stream G, typically via a suitable technical means, preferably via a condenser. It is preferable here to conduct the condensation at a temperature of 80 to 100° C. at a pressure of 2 to 5 hPa, or under appropriate boiling temperature-pressure conditions. Fraction C remains in the gas phase during this condensation.

In one embodiment of the process of the invention, fraction C can be condensed separately at a temperature of −10 to 30° C. at a pressure of 2 to 5 hPa, or under appropriate boiling temperature-pressure conditions. In a further embodiment of the process of the invention, fraction C may remain in the gas phase and be disposed of by incineration, for example.

The process is preferably effected continuously. In this case, the crude product is subjected to continuous thermal treatment, with simultaneous discharge of fraction B from the process in liquid form, and simultaneous production of gas stream G and transport away from the technical means by which it is subjected to thermal treatment, preferably from the fixed heated surface. At the same time, in that case, fraction A and optionally fraction C are condensed. This means that these different operations are in equilibrium during the process of the invention. In this equilibrium, the mass of the crude product added to the process is always essentially identical to the sum total of the masses of fraction A, fraction B and fraction C that are discharged from the process. What is meant by “essentially” in this context is that the fluctuations in mass flow rates for technical reasons per individual unit time are thus included.

A further advantage of a preferred embodiment of the process of the invention is that no entraining agent is added to the crude product at any time in the process. This avoids further wastes. Entraining agents refer to typically liquid substances that are added to the mixture to be distilled, for example to the crude product. These entraining agents generally have a higher boiling point than the components that are separated from the bottoms, for example fraction B, in the form of a gas stream, for example fraction A and/or fraction C, form a homogeneous phase with the mixture to be distilled and are chemically inert, and hence do not react with the components of the mixture to be distilled. An entraining agent generally increases the yield of distillate.

The invention likewise encompasses an apparatus of the invention comprising at least an evaporation unit (1), vessel for fraction B (3), a condenser (5) and a pump (8).

The apparatus of the invention preferably additionally comprises a column (4), a communicating conduit (11) between evaporation unit (1) and column (4), a reflux divider (6), and a condenser (7).

The apparatus of the invention further preferably additionally comprises inlet for crude product (21), communicating conduit (41) between column (4) and condenser (5), communicating conduit (51) between condenser (5) and condenser (7), outlet for fraction C (72), communicating conduit (61) between condenser (5) and reflux divider (6), communicating conduit (62) between column (4) and reflux divider (6), and outlet for fraction A (63).

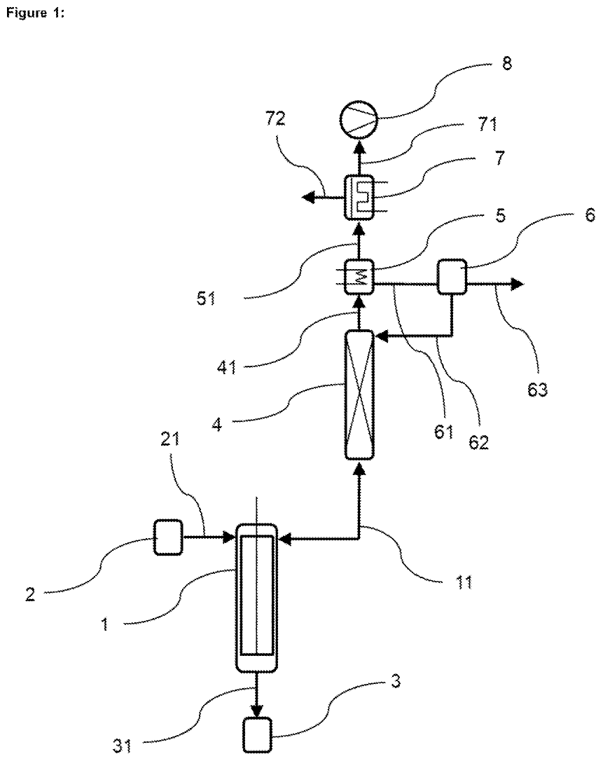

FIG. 1 shows a diagram of a particularly preferred apparatus of the invention.

The labels in FIG. 1 mean:
1 evaporation unit
2 vessel for crude product
3 vessel for fraction B
4 column
5 condenser
6 reflux divider
7 condenser
8 pump
11 outlet for gas stream G, communicating conduit between 1 and 4
21 inlet for crude product
31 outlet for fraction B
41 communicating conduit between 4 and 5
51 communicating conduit between 5 and 7
61 communicating conduit between 5 and 6
62 communicating conduit between 6 and 4
63 outlet for fraction A
71 communicating conduit between 6 and 7
72 outlet for fraction C

In one embodiment, the process of the invention is performed in apparatus comprising at least
an evaporation unit (1), inlet for crude product (21) and outlet for fraction B (31),
   wherein the evaporation unit (1) comprises at least
   a heatable housing shell surrounding a rotationally symmetric evaporation space that extends in axial direction, and
   a drivable rotor shaft extending coaxially within the evaporation space, for production of a crude product film on the inner surface of the housing shell and for conveying of the material in the direction from the inlet for crude product (21) toward the outlet for fraction B (31), where the rotor shaft has a central rotor shaft body and rotor elements disposed on the circumference thereof, the radially outermost end of which is at a distance from the inner surface of the housing shell,
condenser (5), reflux divider (6), outlet for fraction A (63), and preferably column (4),
   wherein, in step a), the crude product is subjected to thermal treatment such that the crude product is introduced via the inlet (21) into the evaporation space of the evaporation unit (1) and a liquid film of the crude product is produced on the inside of the housing shell, as a result of which the liquid film of the crude product is heated to a temperature, such that at least a portion of fraction A and of fraction C is converted to the gaseous state and discharged as gas stream G from the evaporation unit (1), preferably via the outlet (11), where the ratio of mass flow rates between fraction B and gas stream G is from 1:20 to 1:5, and
   in step b), fraction B is discharged via the outlet (31) from the evaporation unit (1), and
   in step c), gas stream G is transferred from the evaporation unit (1), preferably via column (4), into the condenser (5), where fraction A is condensed and obtained via the reflux divider (6) and outlet (63).

The apparatus of the invention preferably additionally comprises a condenser (7) in which fraction C is optionally condensed in step d) and fraction C is optionally discharged via outlet for fraction C (72).

Evaporation unit 1 is preferably a thin-film evaporator or a falling-film evaporator. Evaporation unit 1 is more preferably a thin-film evaporator.

The evaporation unit 1 is typically cylindrical and has an outer housing shell and an inner housing shell. The housing shell may have a single-wall or double-wall design. Advantageously, the housing shell has a double-wall design because of the temperature differential between the inner and outer portions of the evaporation unit 1. It is then possible to install insulation elements and/or heating elements in the interspace, which enables very exact temperature control of the inner face of the housing shell or of the inner housing shell. What is meant by “inner” in this context is always the side of a surface directed toward the center of the cross section of the cylindrical evaporation space. In a coaxial arrangement with respect to the rotationally symmetric evaporation space which is bounded laterally by the inner face of the housing shell is a motor-driven rotor consisting of a rotor axis and rotor elements mounted thereon. The rotor elements extend radially from the center of the rotor axis in the direction of the inner face of the housing shell. There are rigid and dynamic rotor elements.

Rigid rotor elements are, for example, rigid blade rotors, radial wiper rotors or wiper blade rotors. Either an inflexible segment ends here at a short distance from the inner surface of the housing shell or flexible segments, for example movable blades or wipers, are pushed in the direction of the inner surface of the housing shell by the elasticity of the material and/or by the centrifugal force that arises as a result of the rotation of the rotor. In the absence of any product, the ends of these flexible elements would be able to touch the inner surface of the housing shell. In the case of dynamic rotors, for example, moving rollers are used, which are attached to movable arms that are in turn connected again to the rotor axis. The centrifugal force that arises as a result of the rotation of the rotor pushes the rollers in the direction of the inner surface of the housing shell, and would thus be able to touch the inner surface of the housing shell without product being present in the evaporation space. But if a product comprising evaporable components is introduced into the evaporation space, this product is directed by technically suitable means in the direction of the upper portion of the inner surface of the housing shell and is then distributed as a film on the inner surface of the housing shell by the end segments of the rotor either by means of the gap present between the outermost end of a rigid blade or by means of the flexible end segments of the wipers or rollers. The thickness of the film is controlled here by an interplay between at least the viscosity of the product, and the type and speed of the rotor and the rotor elements.

In a preferred embodiment of the process of the invention, the crude product is subjected to thermal treatment in the form of a liquid film having a film thickness of 0.001 to 2 mm, preferably of 0.005 to 1 mm.

In a preferred embodiment of the process of the invention, the crude product is subjected to thermal treatment as a liquid film having a film thickness of 0.001 to 2 mm, preferably of 0.005 to 1 mm, by contacting it with a fixed heated surface.

In a further preferred embodiment of the process of the invention, the crude product is subjected to thermal treatment in the evaporation unit (1) in the form of a liquid film having a film thickness of 0.001 to 2 mm, preferably of 0.005 to 1 mm. In this further preferred embodiment, the inner surface of the housing shell of the evaporation unit (1) forms the heated fixed surface with which the crude product is contacted.

The film thickness results from the technical parameters of the apparatus used for the purpose and from the technical means used. This is described in more detail further down. The liquid film is brought into direct contact with a fixed heated surface. The fixed heated surface may then, for example, be an inner wall of a thin-film evaporator or of a falling-film evaporator. The fixed surface is preferably an inner wall of a thin-film evaporator.

The film thickness is determined, for example, by contacting at a defined flow rate of a specific product at standard pressure with the fixed heated surface at the temperature which is employed in the performance of the preferred process of the invention. What is measured here is the time required for the complete wetting of the fixed heated surface with creation of a film. The product T×R of the measured time T, for example 120 seconds, and the flow rate R, for example 300 cm³/3600 sec., gives the volume V of the film, for example 10 cm³, that has been created over the entire area of the fixed heated surface. Since the area of the fixed heated surface A can be calculated from its geometric data, for example, it is possible to calculate the film thickness F as the quotient V/A, i.e. the volume of the film divided by the area of the fixed heated surface A. In the given example, the area A is 1000 cm² and hence the film thickness is 0.01 cm. Without evaporation of fractions of the product, the average residence time of the product at the fixed heated surface in this example is 120 sec. On the other hand, it is also possible to determine the average residence time when the area A is known and the volume required for the wetting of the total area A at a flow rate R is being measured.

In the preferred process of the invention, the residence time of the liquid components, i.e. crude product and/or fraction B, or any mixtures thereof, at the fixed heated surface is preferably from 1 to 900 seconds, more preferably from 10 to 600 seconds, especially preferably from 10 to 300 seconds.

For the preferred process of the invention, when an evaporation unit (1) is used, for example thin-film evaporators, film thicknesses of 0.001 to 2 mm, preferably of 0.001 to 1 mm, are employed. The film thickness is determined here, for example, by measuring the time required for the complete wetting of the inner surface of the housing shell for creation of a film at a defined flow rate of a specific product which is to be processed in the evaporation unit 1, for example in a thin-film evaporator, at a defined rotor speed and at a defined temperature of the inner surface of the housing shell of the evaporation unit 1, but at standard pressure. The product T×R of the measured time T, for example 50 seconds, and the flow rate R, for example 300 cm³/3600 sec., gives the volume V of the film created, for example 5 cm³, that has been created over the entire inner surface of the housing shell by the rotor. Since the area of the inner surface of the housing shell A can be calculated from its geometric data, for example, it is possible to calculate the film thickness F as the quotient V/A, i.e. the volume of the film divided by the area of the inner surface of the housing shell A. In the example given, the film thickness is 0.05 mm. The average residence time of the liquid phase in the evaporation unit 1 in this example is 60 sec.

In the preferred process of the invention, the residence time of the liquid components, i.e. crude product and/or fraction B, or any mixtures thereof, at the fixed heated surface is from 1 to 900 seconds, preferably from 10 to 600 seconds, especially preferably from 10 to 300 seconds.

In the preferred process of the invention, which uses an evaporation unit 1, the temperature for the inner surface of the housing shell is preferably chosen such that it is not more than 40° C., preferably not more than 30° C., above the boiling point of fraction A at the respective pressure. Likewise preferably, in step a), the gas stream G is converted to the gas phase by contacting with the inner surface of the housing shell of the evaporation unit (1) at a temperature of 120 to 200° C., preferably of 120 to 185° C., more preferably of 120 to 175° C., and a pressure of 1 to 35 hPa, preferably of 1 to 21 hPa, more preferably of 2 to 15 hPa.

The crude product is directed here, preferably through inlet 21, into the upper portion of the evaporation unit 1 via suitable technical means directly onto the upper portion of the heated inner surface of the housing shell of the evaporation unit 1 and begins to flow downward under gravity until the rotating ends of the segments of the rotor elements start to distribute the crude product as a liquid film uniformly on the heated inner surface of the housing shell of the evaporation unit 1. The person skilled in the art will be able here to ascertain the optimal wiping speed of the rotor for the evaporation unit 1 by simple experiments. Too low a wiping speed leads to incomplete distribution of the liquid film and possibly to formation of droplets that flow more quickly through the evaporation unit 1 than the liquid film. Beyond an optimal value for the wiping speed, a further increase in the wiping speed does not bring any further improvement in the liquid film. By virtue of replenishment of crude product, by virtue of gravity and by virtue of the mechanical force of the rotating ends of the rotor elements, the liquid film of the crude product migrates gradually toward the lower end of the heated inner surface of the housing shell of the evaporation unit 1. By virtue of the contact of the liquid film of the crude product with the heated inner surface of the housing shell of the evaporation unit 1, a portion of the crude product is converted to the gas phase as gas stream G, containing fraction A fraction C. Fraction B, rather than being converted to the gas phase at the heated inner surface of the housing shell of the evaporation unit 1, is transported in liquid form under gravity and/or through the movement of the rotor elements continuously in the direction of the base of the evaporation unit 1, where there is outlet 31 for fraction B. Gas stream G, by virtue of the pressure gradient generated by the condensation, is directed in the direction of the upper portion of the evaporation unit 1, where there is outlet 11 for gas stream G. Outlet 11 is preferably in communicating connection with column 4. Column 4 is more preferably a column having column internals, random packings or fixed packings. Preferably, column 4 has from 1 to 20, preferably from 5 to 15 and more preferably from 8 to 12 theoretical plates.

Within column 4, during the preferred process of the invention, there exists a vapor-condensate equilibrium with a concentration gradient in the upward direction. In this concentration gradient, the proportion of higher-boiling components increases toward the base of the column. In this preferred embodiment, a portion of the gas stream G leaves the top of the column 4 continuously in gaseous form and enters the condenser 5 via the conduit 41 which is in communicating connection with condenser 5. Condenser 5 typically has an internal temperature from 50 to 110° C., preferably from 70 to 110° C., such that fraction A condenses, while fraction C leaves the upper portion of the condenser 5 in gaseous form via outlet 51. The condensed fraction A leaves the condenser 5, preferably via outlet 61, which is connected to a conduit, which is preferably in communicating connection with a reflux divider 6. A portion of the condensed fraction A leaves the reflux divider 6 via outlet 63, while a further proportion of the condensed fraction A flows back again to the upper portion of the column under gravity via conduit 62, which is in communicating connection with the upper portion of the column 4.

In a particularly preferred embodiment of the process of the invention, the reflux in the reflux divider 6 is adjusted such that the ratio of the mass flow rates that leave the reflux divider 6 via conduit 62 and conduit 63 is from 1:10 to 1:1, preferably from 1:5 to 1:2. For example, the reflux divider 6, at a mass flow rate of 1:10, is left by ten times the mass of condensed fraction A via conduit 63 compared to the mass of fraction A that leaves the reflux divider 6 via conduit 62, and is returned to column 4. Via conduit 63, in the form of fraction A, the mixture of the invention is obtained, containing from 98.8% to 100.0% by weight, preferably from 99.0% to 99.9% by weight, of compound of the formula (I), from 0.00% to 0.70% by weight, preferably from 0.0001% to 0.20% by weight, of compound of the formula (II) and having a Hazen color number of 0 to 15, measured by the DIN EN ISO 6271 method.

The mass flow rate of condensed fraction A that flows back again into the column via conduit 62 also partly flows back again into the evaporation unit 1.

In a preferred embodiment of the process of the invention, the gas stream G is generated at pressures of 1 to 30 hPa. Gas stream G is typically at a temperature of 170 to 180° C. at a pressure of 20 hPa. At higher pressures and the consequently required higher temperatures for production of the gas stream G, there is an increase in the breakdown of the compound of the formula (I). At the pressures mentioned, the temperature for the inner wall of the evaporation unit 1 is preferably chosen such that it is not more than 40° C., preferably not more than 30° C., above the boiling point of fraction A at the respective pressure. Likewise preferably, the heated fixed surface is heated to temperatures of 120 to 200° C., preferably of 120 to 185° C., more preferably of 120 to 175° C., and a pressure of 1 to 35 hPa, preferably from 1 to 21 hPa, more preferably from 2 to 15 hPa.

The process of the invention is preferably effected continuously. This involves continuously producing a liquid film of the crude product in evaporation unit 1, simultaneously discharging fraction B from the process in liquid form via outlet 31, and simultaneously producing gas stream G via outlet 11 into the column 4 thence transferring it into the condenser 5 via conduit 41.

At the same time, fraction A is then condensed in condenser 5, and fraction C is optionally condensed in condenser 7 and optionally discharged from the process via outlet for fraction C (72). In condenser 7, it is customary to establish an internal temperature of −10 to +30° C., preferably of 0 to +30° C. This means that these different operations are in equilibrium during the process of the invention. In this equilibrium, the mass of the crude product added to the process is always essentially identical to the sum total of the masses of fraction A, fraction B and fraction C that are discharged from the process. What is meant by “essentially” in this context is that the fluctuations in mass flow rates for technical reasons per individual unit time are thus included.

In a particularly preferred embodiment of the process of the invention, the mass flow rate of fraction B is adjusted such that the mass ratio of fraction A which is discharged from the process via conduit 63 to fraction B which is discharged from the process via outlet 31 is continuously from 4:1 to 49:1, preferably from 5:1 to 10:1. Fraction B which is discharged from the process via outlet 31 preferably contains from 40% to 95% by weight of compound of the formula (I), and from 1.5% to 50% by weight of compound of the formula (II).

In a further particularly preferred embodiment of the process of the invention, fraction B is collected and then reintroduced as crude product into the evaporation unit 1, in which case a fraction AA and a fraction BB are then formed. This increases the chemical yield of the mixture of the invention without the composition of the mixture of the invention obtained being outside the claimed limits.

The invention likewise relates to a process for producing the mixtures of the invention in the apparatuses of the invention.

The invention likewise relates to the mixtures obtainable by the process of the invention, containing from 98.8% to 100.0% by weight, preferably from 99.0% to 99.9% by weight, of compound of the formula (I) and from 0.00% to 0.70% by weight, preferably from 0.0001% to 0.20% by weight, of compound of the formula (II) and having a Hazen color number of 0 to 15, measured by the DIN EN ISO 6271 method.

The invention likewise relates to the mixtures obtainable by the process of the invention, containing from 98.8% to 100.0% by weight, preferably from 99.0% to 99.9% by weight, of compound of the formula (I) and from 0.00% to 0.60% by weight, preferably from 0.0001% to 0.20% by weight, of compound of the formula (II) and having a Hazen color number of 0 to 15, measured by the DIN EN ISO 6271 method.

The invention likewise relates to the mixtures obtainable by the process of the invention, containing from 98.8% to 100.0% by weight, preferably from 99.0% to 99.9% by weight, of compound of the formula (I) and from 0.00% to 0.50% by weight, preferably from 0.0001% to 0.20% by weight, of compound of the formula (II) and having a Hazen color number of 0 to 15, measured by the DIN EN ISO 6271 method.

The mixture of the invention preferably additionally contains from 0.0001% to 2.0% by weight of compound of the formula (III). A further advantage of the mixtures of the invention is that they can have a distinctly lower odor than the currently known products that contain more than 90% by weight of compounds of the formula (I). This can be unambiguously shown in double-blind olfactory studies.

By the process of the invention, it is surprisingly possible to overcome the disadvantages of the prior art processes and to obtain the mixtures containing the compound of the formula (I) in an advantageous composition unknown to date. At the same time, the process of the invention, by comparison with the prior art processes, has comparable or higher chemical yields of the compound of the formula (I) and, with avoidance of multiple extractive workup steps for production of the crude product, a higher space-time yield, which makes it possible to perform the process of the invention more economically. The avoidance of multiple extractive workup steps can additionally reduce the amount of waste materials that either have to be incinerated or disposed of in some other way. In the process of the invention, in the mixtures of the invention, i.e. in the isolated fraction A, the compound of the formula (I) is isolated in chemical yields of greater than 95 percent of theory. In addition, in the process of the invention, the proportion by mass of compound of the formula (I) present in fraction A and fraction B, based on the mass of compound of the formula (I) that has been used in the crude product for the production of these fractions A and B, is more than 99 percent. This confirms that virtually no breakdown of the compound of the formula (I) takes place in the process of the invention.

### EXAMPLES

### A. Production of the Crude Product

An empty, inertized 2 m³ Hastelloy C reactor was charged with 125 kg of 100% toluene and, after commencement of stirring at a speed of 60 revolutions per minute, heated to 80° C. Subsequently, 250 kg of water and 481.3 kg (content: 99% by weight, 3.69 kmol) of 2-piperidineethanol (2-(2-hydroxyethyl)piperidine) was added. The feed via which 2-piperidineethanol was transferred into the reactor was rinsed with 50 kg of toluene. While stirring, in parallel, over a period of 6 hours, 453 kg of 32.0 percent sodium hydroxide solution (3.62 kmol) and 500 kg of s-butyl chloroformate (content: 99.0% by weight, 3.62 kmol) were added at 85° C. while maintaining a pH of 8.5 to 9.5. The reaction is exothermic and requires external cooling in order to limit the reaction temperature to 85° C.

After the reaction had ended, the reaction mixture was cooled down to 62° C. 100 kg of 10 percent sulfuric acid (0.102 kmol) was added. The biphasic reaction mixture was stirred for a further 15 minutes. Thereafter, the stirring was ended in order to enable phase separation. After phase separation, 948 kg of lower aqueous phase was discharged from the reactor.

Subsequently, for a first acidic wash, 481.2 kg of toluene (content: 100% by weight), 40 kg of water and 20 kg of 10 percent sulfuric acid (20.4 mol) were added to the organic phase remaining in the reactor. The mixture was heated to 60° C. while stirring and stirred for a further 15 minutes. Again, the stirring was ended in order to enable phase separation. After phase separation, 57.2 kg of lower aqueous phase was discharged from the reactor.

### 1. Crude Product A

3.299 g of the organic phase thus obtained was brought to pH 4.8 to 6 by addition of dilute NaOH, and the aqueous phase was separated off. The organic phase was used for inventive example 4 after the toluene present therein had first been distilled off at 90° C. and 80 hPa. 91 g of water was added to the remaining 1.986 g of bottoms, and 145 g was distilled off at 90° C. and 60 hPa, another 91 g of water was added and distillative removal of 97 g of material left 1.877 g of residue.

The crude product A thus obtained had the following composition:

| Component | Values | |
|---|---|---|
| Compound of formula (I) | 97.9% | by wt. |
| Toluene | 0.4% | by wt. |
| Compound of formula (II) | 0.7% | by wt. |
| Compound of formula (III) | 0.05% | by wt. |

| | |
|---|---|
| Hazen color number | 90 |
| Olfactory assessment | musty odor |

### 2. Crude Product B

Subsequently, the remaining organic phase, from which 3.299 g had been removed for the production of crude product A, for the second acid wash, was once again admixed with 40.0 kg of water and 20.0 kg of 10 percent sulfuric acid (20.4 mol). The mixture was stirred for a further 15 minutes. Again, the stirring was ended in order to enable phase separation. After phase separation, 58.5 kg of lower aqueous phase was discharged from the reactor.

For neutralization, a third wash was included, by adding 125 kg of water to the organic phase remaining in the reactor, in order then to bring the reaction mixture to a pH of 4.8 to 5.0 by adding 0.13 kg of 32% sodium hydroxide solution (1 mol). The mixture was stirred for a further 10 minutes. Again, the stirring was ended in order to enable phase separation. After phase separation, 140.3 kg of lower aqueous phase was discharged from the reactor.

As the last wash, the organic phase remaining in the reactor was mixed with 125 kg of water, where the pH of the mixture was adjusted to a range from 4.8 to 7.0 with 0.13 kg of 32% sodium hydroxide solution (1 mol). The mixture was stirred for a further 10 minutes. Again, the stirring was ended in order to enable phase separation. After phase separation, 137.5 kg of lower aqueous phase was discharged.

Of the remaining organic phase, 641.3 kg of the toluene solvent was distilled off at a pressure of 650 hPa and a bottoms temperature of up to 90° C. Subsequently, 37.5 kg of water was added and the mixture was stirred for a further 60 minutes. Then 40.0 kg of water was distilled out of the bottoms at a maximum of 90° C. and 60 hPa. Subsequently, the pressure in the reactor was increased to ambient pressure with nitrogen, the reactor was cooled to a temperature of less than 60° C., and the crude mixture was discharged from the reactor via a filter.

825.0 kg of crude mixture was obtained, which was used as starting material for the process of the invention.

The crude product B thus obtained had the following composition:

| Component | Values | |
|---|---|---|
| Compound of formula (I) | 98.5% | by wt. |
| Toluene | 0.03% | by wt. |
| Compound of formula (II) | 0.8% | by wt. |
| Compound of formula (III) | 0.1% | by wt. |

| | |
|---|---|
| Hazen color number | 40 |
| Olfactory assessment | slightly musty odor |

B. Process for Producing Mixtures from the Crude Product

### Example 1 (Noninventive)

An apparatus consisting of a 2 L flange vessel with a 10-tray column, dephlegmator with reflux divider and vacuum pump with cold trap was initially charged with 1523 g of crude product B, and the system was evacuated to 20 hPa. The dephlegmator was adjusted to 50° C. and the reflux divided to a reflux ratio (volume ratio of reflux to distillate withdrawal) of 1:1. At internal temperature about 175° C., the system boiled, but the majority of the material condensed in the cold trap. The distillation was then stopped. About 102 g of the material was present in the cold trap and about 1386 g in the bottoms. The material in the cold trap consisted of about 99% of 2-butanol; the bottoms in the flange vessel consisted of about 82.3% by weight of compound of the formula (I) and of 16% of compound of the formula (III) that forms through elimination of butanol from compound of the formula (I). It was thus not possible to obtain a compound of the formula (I) as distillate at 175° C. and 20 hPa. Instead, the product decomposed via elimination of butanol. The residence time of the liquid phase containing a crude product was more than 60 minutes.

### Example 2 (Inventive)

The apparatus used for this example consisted of a thin-film evaporator with oil-filled jacket (length 30 cm, internal diameter 5 cm), 3 wiper arms with rollers and an internal temperature measurement at the bottoms outlet and a thermostated condenser (dephlegmator), vacuum pump with cold trap. The evaporator was heated up with oil at 150 to 160° C. and the vacuum was adjusted to 2 hPa. The condenser was operated with oil at 50° C. 200 to 250 g of crude product B was metered in continuously per hour. The wiper speed was 120 rpm. The internal temperature of the thin-film apparatus was between 135 and 140° C. A total of 5107 g of Saltidin was thus metered in. 4869 g of colorless distillate was obtained as fraction A, 130 g of brown liquid bottoms left the thin-film apparatus as fraction B, and 7.1 g of liquid was collected in the cold trap as fraction C. This corresponds to a mass balance of 98%. The average residence time of the liquid phase containing a crude product and/or fraction B or any mixtures thereof was less than 60 seconds. The product thus obtained had the following properties:

| Component | Values | |
|---|---|---|
| Compound of formula (I) | 98.9% | by wt. |
| Toluene | negligible | |
| Compound of formula ( (II) | 0.7% | by wt. |
| Compound of formula (III) | 0.12% | by wt. |

| | |
|---|---|
| Hazen color number | 5 to 12 |
| Olfactory assessment | little to no odor |
| Yield of theory for fraction A | >95% |

A condensate (condensed fraction C) was collected in the cold trap, which contained 97% by weight of water. The brown to dark brown bottoms contained about 200 to 500 ppm of sodium chloride and sodium sulfate, alongside further traces of inorganic constituents.

### Example 3 (Inventive)

The apparatus used for this example consisted of a thin-film evaporator with oil-filled jacket (length 30 cm, internal diameter 5 cm), 3 wiper arms with rollers and an internal temperature measurement at the bottoms outlet, a 10-tray mirrored trace-heated column filled with a packing of HC4 wire mesh rings (packing length 30 cm, diameter 24 mm, about 10 theoretical plates), a reflux divider and a thermostated condenser (dephlegmator), vacuum pump with cold trap. The evaporator was heated up with oil at 178° C. and the vacuum was adjusted to 3 hPa. The insulated column was externally trace-heated at 164° C. for thermal insulation. The condenser was operated with oil at 90° C. 60 g of crude product B was metered in continuously per hour between the column and thin-film evaporator, with establishment of a reflux ratio of 1:2 (reflux/withdrawal, volume/volume) via the reflux divider. The wiper speed was 120 rpm. The internal temperature of the thin-film apparatus at the lower end was between 155 and 160° C. A pressure of 5.5 hPa was established at the bottom, while a pressure of 3.3 hPa was measured at the top of the column. A total of 1715 g of crude product B was thus metered into the thin-film evaporator. 1687 g of fraction A was obtained as colorless distillate; 17 g of fraction B in the form of brown liquid bottoms left the thin-film apparatus, and 3.8 g of fraction C was condensed in the cold trap. This corresponds to a mass balance of >99% by weight. The average residence time of the liquid phase containing a crude product and/or fraction B or any mixtures thereof was less than 60 seconds. The product thus obtained had the following properties:

| Component | Values | |
|---|---|---|
| Compound of formula (I) | 99.6% | by wt. |
| Toluene | <0.05% | by wt. |
| Compound of formula (II) | <0.005% | by wt. |
| Compound of formula (III) | 0.13% | by wt. |

| | |
|---|---|
| Hazen color number | <10 |
| Olfactory assessment | little to no odor |
| Yield of theory for fraction A | >95% |

A condensate (condensed fraction C) was collected in the cold trap, which contained 90% by weight of 2-butanol, 1.7% by weight of toluene, 1.1% by weight of Saltidin and further constituents. The brown to dark brown bottoms contained 76% by weight of Saltidin, 20.1% by weight of compound of the formula (II) and further constituents.

### Example 4 (Inventive)

The apparatus used for this example consisted of a thin-film evaporator with oil-filled jacket (length 30 cm, internal diameter 5 cm), 3 wiper arms with rollers and an internal temperature measurement at the bottoms outlet, a 10-tray mirrored trace-heated column filled with a packing of HC4 wire mesh rings (packing length 30 cm, diameter 24 mm, about 10 theoretical plates), a reflux divider and a thermostated condenser (dephlegmator), vacuum pump with cold trap. The evaporator was heated up with oil at 178° C. and the vacuum was adjusted to 3 hPa. The insulated column was externally trace-heated at 164° C. for thermal insulation. The condenser was operated with oil at 90° C. 70 g of crude product A was metered in continuously per hour between the column and thin-film evaporator, with establishment of a reflux ratio of 1:2 (reflux/withdrawal, volume/volume) via the reflux divider. The wiper speed was 200 rpm. The internal temperature of the thin-film apparatus at the lower end was between 155 and 160° C. A pressure of 7 hPa was established at the bottom, while a pressure of 3.0 hPa was measured at the top of the column. A total of 1365 g of Saltidin was thus metered in. 1300 g of colorless distillate was obtained, 55 g of brown liquid bottoms left the thin-film apparatus, and 2 g of liquid was collected in the cold trap. This corresponds to a mass balance of >99%. The average residence time of the liquid phase containing a crude product and/or fraction B or any mixtures thereof was less than 60 seconds. The product thus obtained had the following properties:

| Component | Values | |
|---|---|---|
| Icaridin | 99.5% | by wt. |
| Toluene | <0.05% | by wt. |
| Compound of formula (II) | <0.005% | by wt. |
| Compound of formula (III) | 0.07% | by wt. |

| | |
|---|---|
| Color number | <10 |
| Olfactory assessment | little to no odor |
| Yield of theory for fraction A | >95% |

A condensate (condensed fraction C) was collected in the cold trap, which contained 90% by weight of 2-butanol, 1.7% by weight of toluene, 1.1% by weight of Saltidin and further constituents. The brown to dark brown bottoms contained 76% by weight of Saltidin, 20.1% by weight of compound of the formula (II) and further constituents.

1. A mixture comprising, as components, from 98.8% to 100.0% by weight of compound of the formula (I): 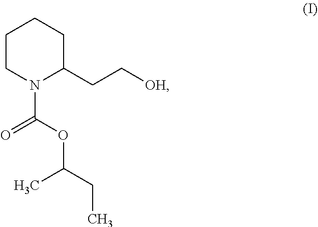 [SMILES: CCC(C)OC(=O)N1CCCCC1CCO]
from 0.00% to 0.60% by weight of compound of the formula (II): 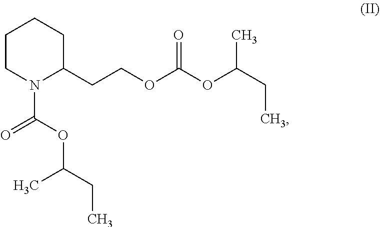 [SMILES: CCC(C)OC(=O)OCCC1CCCCN1C(=O)OC(C)CC]
and further constituents, where the percentages by weight of the components add up to 100% by weight,
wherein the mixture has a Hazen color number of 0 to 15, measured by the DIN EN ISO 6271 method.

2. The mixture as claimed in claim 1, containing from 0.0001% to 2.0% by weight of compound of the formula (III):

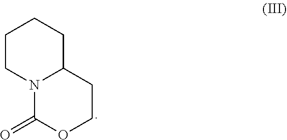 [SMILES: O=C1OCCC2CCCCN12]

3. A process for producing the mixture as claimed in claim 1, proceeding from a crude product containing from 94.0% to 98.0% by weight of the compound of the formula (I),
wherein at least a fraction A (distillate) is separated from a fraction B (bottoms) and a gaseous fraction C, by
a) subjecting the crude product in the form of a liquid phase to thermal treatment such that a gas stream G comprising fraction A and fraction C is produced, and fraction B remains as a liquid phase, and
b) simultaneously and/or successively discharging fraction B from the process in liquid form, and
c) simultaneously and/or successively condensing fraction A out of gas stream G and discharging it from the process, giving the mixture as claimed in claim 1, and
d) optionally simultaneously and/or successively condensing fraction C and discharging it from the process,
wherein a residence time of the liquid phase during the thermal treatment is from 1 to 900 seconds.

4. The process for producing the mixture as claimed in claim 3, proceeding from a crude product having a Hazen color number of at least 40, measured by the DIN-ISO 6271 method.

5. The process as claimed in claim 3, wherein it is conducted in an apparatus comprising at least
an evaporation unit (1), inlet for crude product and outlet for fraction B (31),
wherein the evaporation unit (1) comprises at least
a heatable housing shell surrounding a rotationally symmetric evaporation space that extends in axial direction, and
a drivable rotor shaft extending coaxially within the evaporation space, for production of a crude product film on an inner surface of the housing shell and for conveying of material in a direction from the inlet for crude product toward the outlet for fraction B (31), where the rotor shaft has a central rotor shaft body and rotor elements disposed on the circumference thereof, a radially outermost end of which is at a distance from the inner surface of the housing shell,
condenser (5), reflux divider (6), and outlet for fraction A (63) (4),
wherein, in step a), the crude product is subjected to thermal treatment such that the crude product is introduced via the inlet (21) into the evaporation space of the evaporation unit (1) and a liquid film of the crude product is produced on the inside of the housing shell, as a result of which the liquid film of the crude product is heated to a temperature, such that at least a portion of fraction A and of fraction C is converted to the gaseous state and discharged as gas stream G from the evaporation unit (1), where the ratio of the mass flow rates between fraction B and gas stream G is from 1:20 to 1:5, and
in step b), fraction B is discharged via the outlet (31) from the evaporation unit (1), and
in step c), gas stream G is transferred from the evaporation unit (1) into the condenser (5), where fraction A is condensed and obtained via the reflux divider (6) and the outlet (63).

6. The process as claimed in claim 5, wherein it is conducted in an apparatus comprising a condenser (7) in which fraction C is condensed in step d).

7. The process as claimed in claim 5, wherein the liquid film which is subjected to thermal treatment in the evaporation unit (1) has a film thickness of 0.001 to 2 mm.

8. The process as claimed in claim 3, wherein, in step a), fraction A is converted to the gas phase
at a temperature of 120 to 200° C. and a pressure of 1 to 35 hPa, or
at a temperature not more than 40° C. above the boiling point of fraction A.

9. The process for production as claimed in claim 3, wherein no entraining agent is added to the crude product at any time in the process.

10. The process as claimed in claim 3, wherein the crude product is generated by the steps comprising
i. reacting 2-hydroxyethylpiperidine with sec-butyl chloroformate in the presence or absence of at least one base and in the presence or absence of at least one solvent, giving a reaction mixture containing from 35% to 65% by weight of compound of the formula (I), and
ii. subsequently, optionally while mixing, adding acid and/or water to the reaction mixture from step i., forming a biphasic reaction mixture comprising an organic phase and an aqueous phase, and
iii. subsequently, optionally separating the organic phase from the aqueous phase of the reaction mixture from step ii.;
iv. subsequently, optionally washing the organic phase from step iii. with aqueous acid;
v. subsequently, optionally drying the organic phase from step iv.;
vi. subsequently, optionally separating the at least one solvent from the organic phase from step v., giving the crude product.

11. An apparatus comprising at least an evaporation unit (1), vessel for fraction B (3), a condenser (5) and a pump (8).

12. The apparatus as claimed in claim 11, additionally comprising a column (4), a communicating conduit (11) between evaporation unit (1) and column (4), a reflux divider (6), and a condenser (7).

13. The apparatus as claimed in claim 12, additionally comprising inlet for crude product (21), communicating conduit (41) between column (4) and condenser (5), communicating conduit (51) between condenser (5) and condenser (7), communicating conduit (61) between condenser (5) and reflux divider (6), communicating conduit (62) between column (4) and reflux divider (6), outlet for fraction A (63) and outlet for fraction C (72).

14. The process as claimed in claim 3 for production of the mixture as claimed in claim 1, wherein the process uses the apparatus as claimed in claim 11.
